(19) United States
(12) Patent Application Publication
(10) Pub. No.: US 2008/0147143 A1
(43) Pub. Date: Jun. 19, 2008

(54) DEVICE FOR THE TRANSCUTANEOUS ELECTRICAL STIMULATION OF MOTOR NERVES AND/OR SENSORY NERVES

(75) Inventors: Dejan Popovic, Belgrade (YU); Christian Rohrer, Linz (AT)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: Lohmann & Rauscher GmbH, Schonau / Triesting (AT)

(21) Appl. No.: 11/954,157

(22) Filed: Dec. 11, 2007

(30) Foreign Application Priority Data
Dec. 11, 2006 (DE) 102006058346.9

(51) Int. Cl. A61N 1/04

(52) U.S. Cl. 607/48

(57) ABSTRACT

The invention relates to a device for the transcutaneous electrical stimulation of motor nerves and/or sensory nerves, having an electrode assembly which can be attached to the human body, for example, in the area of the back muscles and/or abdominal muscles, and a stimulation device capable of being operated to apply an electric current to the electrode assembly, with there being at least one motion sensor assembly, operable to detect body movements, associated with the stimulation device and with the stimulation device for the application of the electric current being capable of being operated as a function of the motion signals issued by the motion sensor assembly.

### CROSS REFERENCE TO RELATED APPLICATION

The present application claims international priority under 35 U.S.C. § 119 to co-pending German Patent Application No. 102006058346.9 filed Dec. 11, 2006, entitled “Vorrichtung zur Transkutanen Electrischen Stimulation Motorischer und/oder Sensorischer Nerven,” the entire content and disclosure of which is hereby incorporated by reference in its entirety.

### TECHNICAL FIELD

The invention relates to a device for the transcutaneous electrical stimulation of motor nerves—and/or sensory nerves, having an electrode assembly which can be attached to the human body, for example, in the area of the back muscles and/or abdominal muscles, and a stimulation device capable of being operated to apply an electric current to the electrode assembly, as well as a method for the transcutaneous electrical stimulation of motor nerves and/or sensory nerves using such a device.

### BACKGROUND

Devices of the type described in the introduction are used, for example, for the focused development of the back muscles and/or abdominal muscles. This makes it possible, among other things, to support the spine in the lumbar region. In this context, a conductive gel is usually placed between the electrode assembly and the skin in order to improve the stimulation of the motor nerves, causing muscle activation, by means of the electric current applied. In this process, it is possible for problems to arise during the selective application of the electric current in the area of the motor nerves if the conductive gel is distributed over an area that is too large.

In view of this deficiency, U.S. Pat. No. 4,919,148 proposed an improvement of known devices of the type described in the introduction, inhibiting the spread of the conductive gel by means of a textile structure supporting the electrode assembly. Further devices for the transcutaneous and/or percutaneous electrical stimulation of motor nerves and/or sensory nerves are described, for example, in U.S. Pat. No. 6,445,955 B1, CA 2 496 879 A1, DE 103 00 069 A1, DE 102 48 235 A1, DE 101 55 551 A1, EP 0 052 087 A1, DE 601 11 388 T2, EP 1 144 045 B1, DE 295 22 012 U1 and DE 202 00 685 U1. In addition, it has also been suggested to control the nerve stimulation depending on the body posture and/or, for example, the electromyographically determined muscle activity. Respective devices are specified in EP 1 121 956 A1, U.S. Pat. No. 6,341,237 B1, U.S. Pat. No. 5,643,329, DE 35 16 279 A1 and DE 20 2006 008 616 U1. And finally, EP 1 324 403 B1 describes the use of electro-active elastic actuators for the application of movements of the human body.

However, it has been shown that the use of the devices described in the above listed patent specifications also frequently leads to pain in the lumbar region despite the development of the muscles therewith.

### BRIEF DESCRIPTION OF THE DRAWINGS

Embodiments of the invention are explained below with reference to the drawings, to which reference is made with respect to all details that are material to the embodiments.

FIG. 1 is a schematic representation of a device in accordance with the invention;

FIG. 2 is a representation of an electrode assembly attached to a belt;

FIG. 3 is a representation illustrating the selective control of individual electrode elements of an electrode assembly in accordance with the invention; and

FIG. 4 includes schematic representations to illustrate desirable and harmful movement patterns.

### DETAILED DESCRIPTION OF EMBODIMENTS OF THE INVENTION

In view of the problems in the state of the art, the invention is based on the technical problem of providing a device of the type described in the introduction, enabling the muscle development while simultaneously promoting painless or harmless movements.

In accordance with a first aspect of the invention, this task is solved by a further development of the known devices, characterized essentially in that there is at least one motion sensor assembly, operable to detect body movements, associated with the stimulation device and that the stimulation device for the application of the electric current is capable of being operated as a function of the motion signals issued by the motion sensor assembly. In this arrangement, the motion sensor assembly may include sensors which can be attached to the body, with said sensors possibly being spatially separated on the body from the electrodes of the electrode assembly. It is conceivable for the motion sensors to be arranged on the abdomen and the electrodes to be arranged on the back.

This invention is based on the insight that the problems observed during the use of the known devices are due, first and foremost, to the fact that, despite the regional containment of the application of the electric current to the human body, electrical stimulation of motor nerves may lead to incorrect movements, which could lead to painful states in the lumbar region, with it not being possible to eliminate this deficiency, even by the use of stimulation as a function of the muscle activity detected with the suitable sensors or the body posture. In accordance with the invention, this undesirable result is prevented by monitoring not only the muscle activity or the body posture, but also the body movements by means of a suitable motion sensor assembly, and by a preset current application taking place in the event of unfavorable or harmful movements. In this way, due to the preset current application, it is either possible for the muscles of the human body to be directly activated in such a way that the unfavorable movement is automatically counteracted, or it is possible, due to the current application, for a signal to be given out which is perceivable, if necessary, only subconsciously, and which causes the user of a device in accordance with the invention to terminate the unfavorable movement.

Surprisingly, it has been shown in both cases that, given regular use of devices in accordance with the invention, a learning effect is produced, which has the effect that the movements causing states of pain are permanently suppressed.

Expediently, the stimulation feature of a device in accordance with the invention includes a storage assembly in which data representing preset movement patterns is stored. This makes it possible to compare the body movements captured by the motion sensor assembly with the preset movement patterns, and, in the event of deviations from the preset movement patterns, the muscle activity can be influenced by the respective control of the electrode assembly so that the preset movement pattern is reestablished.

The motion sensor assembly can include one, two or more sensor elements arranged on the surface of the body, with this sensor system allowing the detection of characteristic changes preferably for trunk rotations with unmoved legs, trunk bending in a physiologically risky way, bending to one side or the like. Expediently, the sensor system also allows the detection of change speeds with respect to the distance and/or alignment of two sensor elements on the surface of the body.

In an advantageous embodiment of the invention, the sensor system includes two solid-state measurement devices for axial acceleration. It is not, however, restricted to this technology, but it can also include optical fibers, optical transmitters, ultrasound transmitters, flexible substrates with measurement devices for mechanical tensions or other measurement devices. The motion sensor assemblies which can be used within the framework of the invention are described, for example, in DE 42 05 790 A1. The disclosure content of this patent specification is hereby included in this description by explicit reference with respect to the structure and function of motion sensor assemblies for the detection of movements of the human body.

The use of a motion sensor assembly has been shown to be especially expedient within the framework of the invention for the additional reason that the chronological sequence and the level of muscle activation designed to achieve the desired results can change in terms of time and during a movement.

In accordance with a further aspect of the invention, a device in accordance with the invention for the transcutaneous electrical stimulation of motor nerves, having an electrode assembly which can be attached to the human body, for example, in the area of the back muscles and/or abdominal muscles, and a stimulation device capable of being operated to apply an electric current to the electrode assembly, displays an electrode assembly with four or more pairs of electrodes that are capably of being supplied with electric current independently of each other. Using such an assembly, four or more muscle groups can be activated independently of each other so that the preset movement pattern can be reproduced especially precisely. Using four or more independently controllable pairs of electrodes, it is possible to stimulate two or more pairs of muscle groups belonging together (for example, agonist and antagonist muscles) in order to recreate functional movements particularly well. The function attained in this way is a very important element of rehabilitation because it leads to a more expeditious and more effective cortical reorganization. In this context, it has been shown to be particularly expedient if the electrode assembly includes six or more, specifically eight or more, pairs of electrodes pairs that are capable of being supplied with electric current independently of each other. In this context, it is possible for the stimulation device to include a control device, allowing for the independent control of each of the electrode pairs. This control device can control the electrode pairs, preferably as a function of the movement signals produced by means of the motion sensor assembly.

In addition, or in the alternative, it is also conceivable for the devices to be such that the electrodes are controlled in accordance with preset programs in such a way that the preset movement patterns are reproduced. Within the framework of the invention, it is especially expedient and preferred in order to achieve the preset movement pattern if the electrode pairs are capable of being controlled independently of each other in terms of the stimulation frequency and/or the stimulation amplitude.

In accordance with a third aspect of the invention, the electrode assembly includes at least one pair of electrodes, having a first electrode designed to apply the electric current to the human body via a first contact surface, and having a second electrode designed to discharge the electric current from the human body via a second contact surface, with the size, form and/or position of at least one contact surface being modifiable following placement of the electrode pair on the human body. Using such an electrode assembly, it is possible to adapt the introduction of the current pulses, which cause the muscle activation, particularly well to the particular characteristics of the anatomy of the user of a device in accordance with the invention, if the size, form and position of the contact surface of at least one electrode is adapted such that the current application occurs precisely above the nerves to be stimulated. In addition, it also is possible to adjust the current strength of the introduced current pulses via adjustment of the size of the contact surface. Using a device in accordance with the invention, these adjustments can be made after the placement of the electrode assembly on the human body so that the inconvenient detachment and reattachment of individual electrodes is eliminated.

The adaptation of the size, form and position of the contact surfaces of the electrodes to the particular characteristics of the anatomy of the user of devices in accordance with the invention can occur in a particularly simple way if at least one electrode includes a field of electrodes having a number of electrode elements that are electrically isolated and that are capable of being controlled individually or in groups separately from each other and that have a respective contact surface area of 1 cm² or more. Using such an electrode designed as an electrode field, it is possible to adapt the introduction of the current pulses causing the muscle activation particularly precisely to the particular characteristics of the anatomy of the user of a device in accordance with the invention, if only those electrode elements that are arranged directly above the nerves to be stimulated are controlled. In this context, it is also possible, with the electrode designed as a field of electrodes, to adjust the size and form of the activated electrode as well as the current strength of the introduced current pulses by controlling the electrode elements accordingly.

The electrode elements of the field of electrodes may be arranged in the form of a matrix, in particular in the form of a rectangular matrix, with a multitude of electrode element rows, running parallel to each other, and a multitude of electrode element columns, running vertical to the rows of electrode elements.

Apart from the rectangular form, it is also possible to select other forms, such as, for example, the form of a hexagon, circle or the like within the framework of the invention.

For the selection of the current strength by the selective control of individual electrode elements, the current strength required to activate the motor system is to be taken into account as the lower limit. The pain threshold is to be taken into account as the upper limit.

The electrode assembly of the device in accordance with the invention is capable of being attached to the human body in a particularly simple way if the electrode elements of an electrode are attached to a common carrier made of an electrically isolating material. This carrier is expediently a web-shaped carrier made from a textile material, preferably with each electrode having its own carrier attached, which enables the placement of the respective electrode independently of the placement of the other electrodes. Within the context of the invention, it is also conceivable for the carriers of the electrodes to be attached to a main carrier, for example by means of a Velcro (hook and loop) fastener and to attach it to the body by means of this main carrier. In this context, the main carrier may be implemented, for example, in the form of a belt made from a flexible and/or textile material.

The fields of electrodes of the device in accordance with the invention form the interface at which the electrical charge from the stimulation device may be applied to the skin or discharged from the skin, and said fields of electrodes are preferably made from a textile material or a different material, allowing the electric current to be evenly supplied, in accordance with a preset program, across the field of electrodes, resulting in a muscle contraction. Expediently, the stimulation device is designed as a multi-channel device with galvanic isolation between the individual channels.

The stimulation device preferably comprises signal connecting elements between the electronic stimulation assembly and control devices for the fields of electrodes, a command interface allowing the selection of the program for activation of the stimulation, leading to the electrical activation of the muscles with the device in accordance with the invention, an interface with the wireless programming of a suitable computer system (PC or PDA), an input for sensor signals for the electrical stimulation triggered by the sensor signals, which triggers stimulation sequences to suitable muscle groups in the event that a movement is detected which might lead to back pain. Using the stimulation device of a device in accordance with the invention, the electric current may be distributed preferably in an intermittent, continuous or modulated fashion with low or high frequencies to four, six, eight or more channels capable of being electrically operated independently of each other. Expediently, a bipolar stimulation is used. Using the stimulation device, the amount of electric charge per second is expediently adjusted by optionally changing the pulse duration, the pulse intensity or the pulse frequency.

In an especially preferred embodiment of the invention, the stimulation device for the supply of electric current in the form of current pulses is capable of being operated at a frequency in the range of 1 to 100 kHz, particularly 1 to 100 Hz, with a frequency in the range of 20 to 50 Hz being preferred.

With a stimulation frequency of 16 pulses per second or less, an unpleasant tremble is produced, while a stimulation frequency of higher frequencies produces a muscle tensioning effect. The higher the stimulation frequency, the more pleasant the treatment is for the user of the device in accordance with the invention. At the present time, a device in accordance with the invention is especially preferably operated at a stimulation frequency of 50 pulses per second. The effects of stimulation at over 100 Hz are no different than the effects of stimulation at 100 Hz.

As previously mentioned, it is particularly expedient if the frequency and/or the current strength and/or the phase and/or the voltage of the current pulses is/are capable of being set individually or in groups separately from each other for at least two, preferably at least three, specifically four or more pairs of electrodes of the electrode assembly. In this context, the current strength may be adjusted by the selective control of individual electrode elements and/or individual groups of the electrode elements of a field of electrodes.

Via the phase, in particular, the pulses may be coordinated and/or synchronized with each other, for example, in an intentional chaotic way or in patterns. In view of the convenient handling of the device in accordance with the invention, the electrode assembly is attached to a belt, preferably in a detachable manner, with said belt being capable of being attached to the human body in such a way that it stretches around the body in the lumbar region and with the electrode assembly being arranged between the belt and the body. In this context, the individual electrodes of the electrode assembly may have an isolated design on the side facing the belt.

As can be gathered from the above explanation of devices in accordance with the invention, a method for the transcutaneous electrical stimulation of motor nerves which can be implemented using said device, involving the supply of an electric current to the human body in the area of the back muscles and/or the abdominal muscles via an electrode assembly, is characterized in that the electric current is applied to the electrode assembly as a function of the motion signals, representing body movements, of a motion sensor assembly and/or characterized in that four or more muscle groups are activated by four or more pairs of electrodes independently of each other, with the form and size of the locations of current application expediently being selected by the respective control of individual electrode elements of an electrode designed as a field of electrodes.

In the following, the invention is explained with reference to the drawings, which are explicitly referred to with respect to all particulars essential to the invention and not set forth in detail in the description.

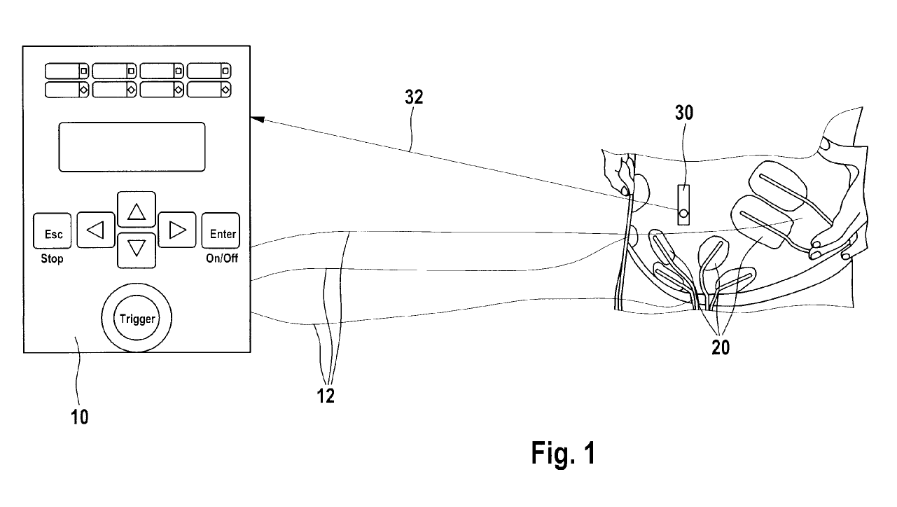

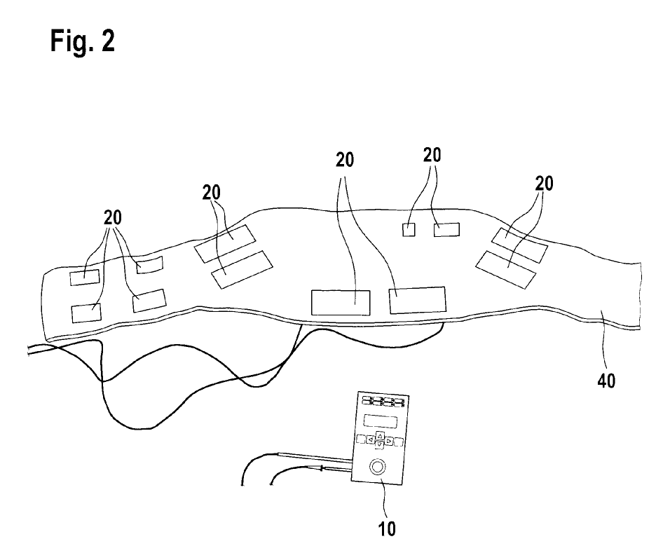

The device shown in FIG. 1 consists essentially of a control device 10, a multitude of electrode pairs 20 attached to a belt 40 (cf. FIG. 2) and a sensor element 30. As shown in FIG. 1, the electrode pairs 20 are controlled via a cable 12, starting from the control device 10. The movements triggered thereby or otherwise are detected by a motion sensor assembly 30, which is connected to the control device 10 via a cable 32, in order to achieve the adjustment of the electrode control using the movements effected thereby as a control variable.

As can be gathered from FIG. 2, the electrode pairs 20 are attached to the inner side, facing the body, of a belt 40.

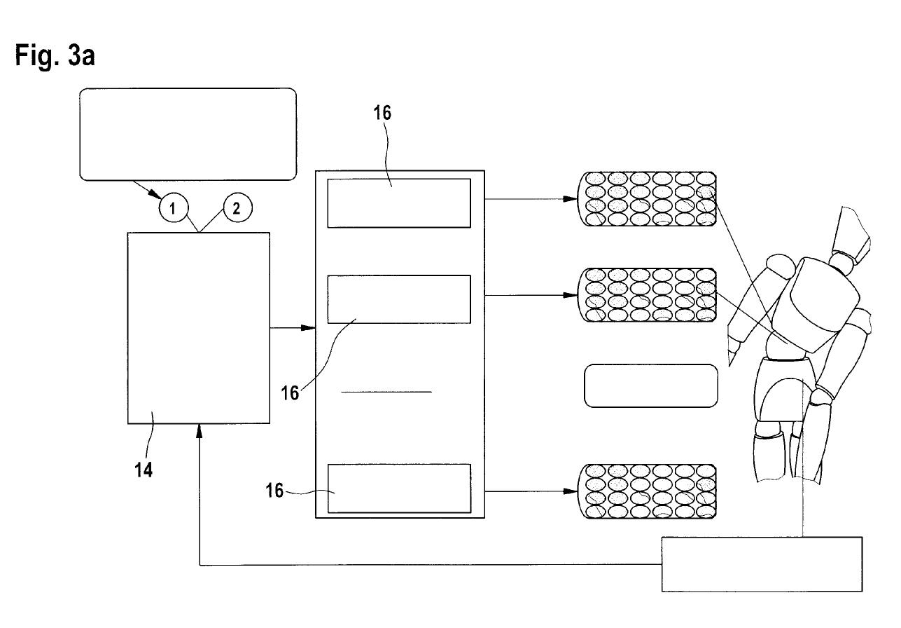

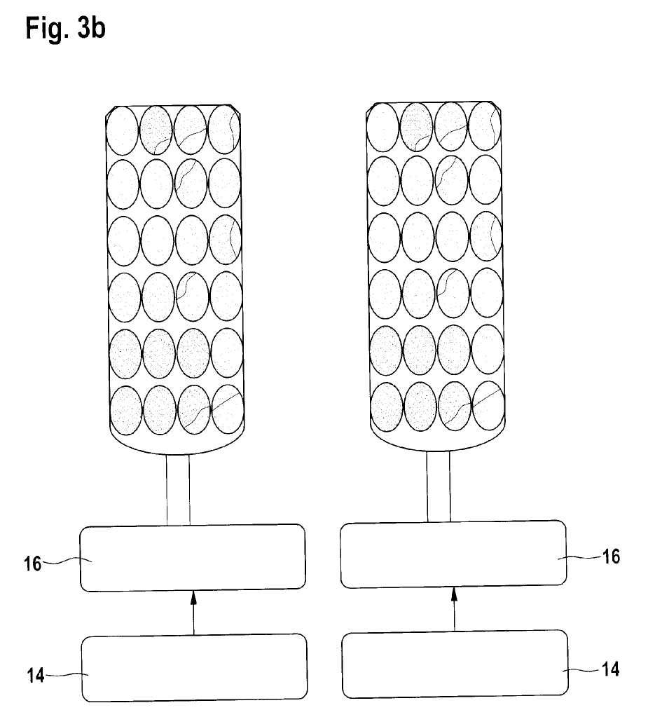

In accordance with FIG. 3, the control device 10 comprises a multi-channel stimulation device 14 for the selective and independent control of individual pairs of electrodes as well as a multitude of electrode control devices 16. By means of the multi channel stimulation device, the stimulation frequency is selectively set for each individual channel, while the current strength is selectively set for each electrode pair by means of the electrode control devices in such a way that individual electrode elements of the electrode designed in the form of fields of electrodes are controlled.

As shown especially clearly in FIG. 3b, the fields of electrodes are arranged in the form of a rectangular matrix with a multitude of rows of electrode elements (6 in the example shown) and columns of electrode elements (4 in the example shown), with the electrode elements of each electrode being fastened to a common carrier which is capable of being positioned independently of the carriers of the other electrodes. The selective control of individual electrode elements of the fields of electrodes makes it possible to selectively set the current strength for the individual electrode pairs, with this also enabling the setting of the place of stimulation by means of a suitable selection of the electrode elements controlled. In contrast to the example shown, the electrode control device may also be integrated into the fields of electrodes or integrated into the stimulation device 14.

The devices shown in the drawing enable the control of the individual pairs of electrodes based on preset programs. In this context, the electrode control device 16 furnishes pulse sequences that are selectively adjustable for each pair of electrodes.

In accordance with a different mode of operation, the pairs of electrodes may be controlled on the basis of the movements of the user that are detected by the motion sensor assembly 30. In this process, harmful movements are detected by the motion sensor assembly 30 and specific muscle groups are activated such that the harmful movements are counteracted.

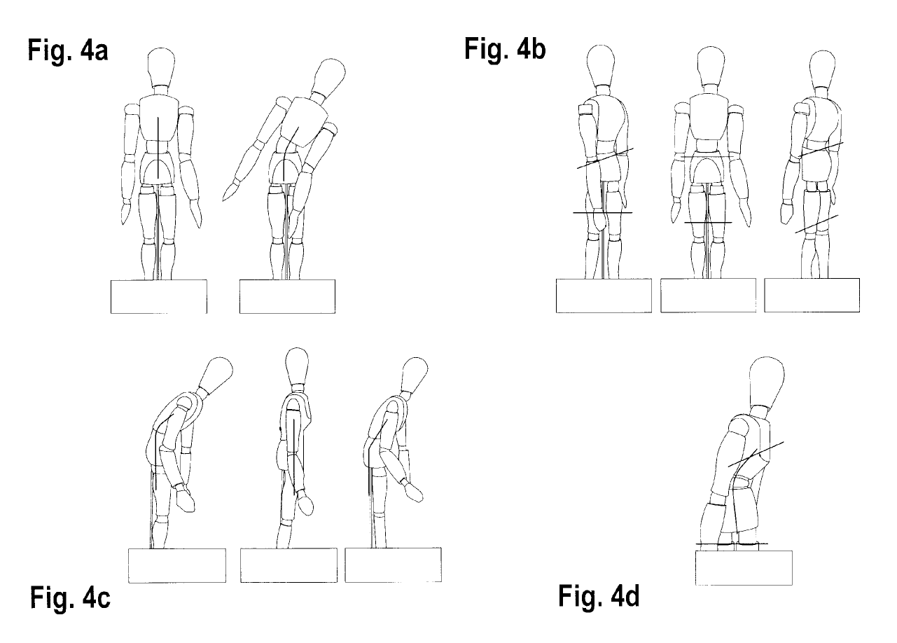

In accordance with FIG. 4a, for example, a lateral bending movement of the upper body can be detected by the motion sensor assembly 30 as a harmful movement, which is shown on the right side in FIG. 4a. The correct posture is shown on the left side in FIG. 4a.

In accordance with FIG. 4b, it is also possible to detect a torsion of the trunk, shown on the left side in FIG. 4b, by the motion sensor assembly 30, and individual muscle groups can be controlled such that, instead of the torsion, a rotary motion of the lower limbs is produced, as shown in the middle and on the right side in FIG. 4b.

As shown in FIG. 4c, it is also possible for a harmful trunk movement in the lumbar region to be detected by the motion sensor assembly 30 and the control of individual muscle groups to take place such that the bending in the lumbar region is replaced by a bending in the hip region. In addition, the motion sensor assembly 30 also makes it possible to identify the combined bending and turning motion shown in FIG. 4d as a harmful movement and to control the muscle groups in such a way that this motion is avoided.

The devices and methods in accordance with the invention make it possible not only to achieve the focused development of muscles, but also the treatment of back pain. In addition, movement training can be implemented such that movements causing back pain are reduced. In addition, the application of devices and methods in accordance with the invention is not restricted to the area of the back muscles and abdominal muscles. It is also possible to treat any other muscle groups.

1-14. (canceled)

15. A device for transcutaneous electrical stimulation of motor nerves and/or sensory nerves, comprising:
an electrode assembly configured to attach to a human body; and
a stimulation device configured to apply an electric current to the electrode assembly;
wherein the stimulation device includes at least one motion sensor assembly configured to detect movements of the human body, and wherein the stimulation device is configured to apply the electric current as a function of motion signals issued by the motion sensor assembly.

16. The device of claim 15, wherein the electrode assembly is configured to attach to at least one of back muscles and abdominal muscles.

17. The device of claim 15, wherein the stimulation device is configured to be operated with a preset movement pattern for comparing the movements detected by the at least one motion sensor assembly.

18. The device of claim 15, wherein the electrode assembly includes four or more pairs of electrodes configured to be supplied with the electric current independently of each other.

19. The device of claim 15, wherein the electrode assembly includes at least one pair of electrodes, including a first electrode configured to apply the electric current to the human body via a first contact surface, and a second electrode configured to discharge the electric current from the human body via a second contact surface.

20. The device of claim 19, wherein at least one of the first contact surface and the second contact surface has one or more of a size, a form, or a position configured to be modified on placement of the at least one pair of electrodes on the human body.

21. The device of claim 19, wherein at least one electrode includes a field of electrodes including a plurality of electrode elements electrically isolated from each other and having a contact surface area of 1 mm² or more, wherein the stimulation device is configured to control the plurality of electrode elements individually or in groups.

22. The device of claim 21, wherein an area of the contact surface of the field of electrodes is 1 cm² or more.

23. The device of claim 21, wherein the electrode elements are arranged in a form of a matrix including a plurality of rows running approximately parallel to each other and a plurality of columns running approximately vertically to the rows.

24. The device of claim 21, wherein the electrode elements of the at least one electrode are attached to a common carrier comprising an electrically isolating material.

25. The device of claim 24, wherein the common carrier is web-shaped.

26. The device of claim 15, wherein the stimulation device is configured to apply the electric current as current pulses, and wherein the stimulation device is configured to deliver the current pulses at a frequency in a range of 1 to 100 kHz.

27. The device of claim 26, wherein the stimulation device is configured to deliver the current pulses at a frequency in a range of 1 to 100 Hz.

28. The device of claim 27, wherein the stimulation device is configured to deliver the current pulses at a frequency in a range of 20 to 50 Hz.

29. The device of claim 26, wherein the stimulation device is configured to control one or more of a frequency, a current strength, a phase, or the voltage of the current pulses.

30. The device of claim 29, wherein the electrode assembly includes a plurality of pairs of electrodes configured to apply the current pulses to the human body, and wherein the stimulation device is configured to control at least two pairs of the electrodes individually or in groups.

31. The device of claim 26, wherein the stimulation device is configured to coordinate the current pulses with each other.

32. The device of claim 26, wherein the stimulation device is configured to synchronize the current pulses with each other.

33. The device of claim 15, wherein the electrode assembly includes at least one field of electrodes including a plurality of electrode elements, and wherein the stimulation device is configured to adjust a density of the current pulses by selectively controlling the plurality of electrode elements individually or in groups.

34. The device of claim 15, further comprising a belt being configured to attach to the human body in a manner extending around a lumbar region of the human body, wherein the electrode assembly is configured to attach to the belt between the belt and the human body.

35. The device of claim 34, wherein the electrode assembly is configured to detachably attach to the belt.

36. A method for transcutaneous electrical stimulation of motor nerves and/or sensory nerves, comprising:
detecting movements of a human body;
issuing motion signals based at least in part on the movements; and
applying an electric current to the human body as a function of the motion signals.

37. The method of claim 36, wherein the electric current is applied to at least one of back muscles and abdominal muscles.
